(19) United States
(12) Patent Application Publication
(10) Pub. No.: US 2022/0154150 A1
(43) Pub. Date: May 19, 2022

(54) ARTIFICIAL VIRUS PRESENTING CELLS

(71) Applicant: Diagnologix, LLC, San Diego, CA (US)

(72) Inventors: Yu-Tsueng LIU, San Diego, CA (US); Guixin SHI, San Diego, CA (US); Chuntang FU, San Diego, CA (US)

(21) Appl. No.: 17/550,958

(22) Filed: Dec. 14, 2021

Related U.S. Application Data
(63) Continuation of application No. PCT/US2021/058634, filed on Nov. 9, 2021.
(60) Provisional application No. 63/113,817, filed on Nov. 13, 2020.

(51) Int. Cl. C12N 7/00; C12N 15/85; A61K 35/76

(52) U.S. Cl. CPC C12N 7/00; C12N 15/85; A61K 35/76; C07K 16/2809; C12N 2710/22023; C12N 2710/22051; C12N 2710/22022

(57) ABSTRACT

A method for ex vivo transduction of biomolecules from viruses, viral vectors or virus-like particles into target cells and microbubbles for use in this method. A quantity of viruses, viral vectors or virus-like particles and target cells are bound to flexible lipid shell microbubbles, bringing these into close proximity to each other that allows viral transduction, transferring biomolecules from the viruses, viral vectors or virus-like particles into the target cells while the viruses, viral vectors or virus-like particles and the target cells are bound to the microbubbles.

### FIELD OF THE INVENTION

The present disclosure relates to flexible lipid shell microbubbles adapted to facilitate viral transduction between viruses, viral vectors or virus-like particles and target cells and a method for ex vivo transduction of biomolecules based on the aforementioned flexible lipid shell microbubbles.

### BACKGROUND OF THE INVENTION

The cell processing industry has manufacturing bottlenecks and is working on system automation for CAR-T cell production. The major steps include T cell selection and activation, gene transfer (currently mainly through viral transduction), and cell expansion and formulation. For example, the Prodigy (Miltenyi) is an all-in-one functionally closed processing machine created by assembling familiar instruments that are routinely used in a cell biology laboratory. Conceptually, it is a miniaturized laboratory and requires various setups for each step of its continuous processing, which may substantially drive up the costs.

From the U.S. Pat. No. 10,479,976 targeted microbubble-based technologies for cell sorting and ligands presentation is known, improving quality of processed cells and reducing manufacturing costs by producing Tscm (central memory T stem) cell-based CAR-T cells. Lower cell dose was sufficient with more potent Tscm cells, leading to cost saving. This is mainly due to the lower number of viruses needed for transduction, which has been a major cost in current mainstream CAR-T manufacturing.

Cell culture automation is a relatively mature area in the cell processing industry and nearly every marketed system can be a standalone unit, regardless of what the upstream system is. Recreating a new large-scale cell culture module would not be cost-effective and could also be counterproductive, as the cell expansion process demands a much longer time (days to weeks) than the first 3 steps combined. An all-in-one device cannot be available to process the next sample until the cell culture is completed.

From the U.S. Pat. No. 10,479,976 targeted microbubble-based technologies are known for ex vivo bulk-volume cell isolation (BUBLES: buoyancy enabled separation) and cell surface agent presentation by applying several interesting properties of microbubbles. From the U.S. Pat. No. 10,479,976 a multiparametic bulk-volume cell-sorting platform is a known, designated “iterative BUBLES” (iBUBLES), that can utilize any off-the-shelf antibodies without the need for reengineering. It takes advantage of the disruptable property of microbubbles, which is not possible for solid magnetic particles, so that a series of antibody conjugated MBs can be applied to isolate specific subset of cells. Moreover, the entire procedure can be performed in a single, syringe-like container.

Also known from the U.S. Pat. No. 10,479,976 is a second step of CAR-T cell processing by applying anti-CD3/CD28 conjugated microbubbles (MB-anti-CD3/CD28) as artificial antigen presenting cells (aAPCs) for robust T cell activation and expansion. These MB-anti-CD3/CD28, in comparison to the commercial gold standard, offer superior long-term expansion of primary human T cells ex vivo. Notably, this is a “bead-free” method, as microbubbles spontaneously burst within 24 hours when cells are grown under standard culture conditions.

From the U.S. Pat. Nos. 5,686,278, 6,033,907, 7,083,979, and 6,670,177 a recombinant human fibronectin fragment known under the trade name retronectin (CH-296) is known which increases the efficiency of retrovirus mediated gene transfer. Further, from the international patent application publication WO 2010080032 A2 a bead-assisted viral transduction is known.

Retroviral Transduction

T cell and aAPC (MB-anti-CD3/CD28) interaction leads to immunological synapse formation. Similarly, “virological synapse” formation has been described as an efficient mechanism for direct cell-to-cell transmission by retroviruses, including HIV-1 and HTLV-1. In fact, in vitro cell-to-cell transfer of HIV-1 between T cells is much more efficient (100-1000 fold) than infection arising from cell-free viral particles. Here we disclose various types of microbubble-based artificial virus-presenting cells (aVPC's) that display viral particles along with cell targeted ligands on fluid lipid surface to recapitulate cell-to-cell viral transmission.

While cell entry for retroviruses requires specific receptors interaction, the initial viral attachment onto cells is not a specific receptor binding event. It needs to overcome electrostatic repulsion between the negatively charged cells and enveloped viruses. Many chemical and physical methods have been developed to enhance viral transduction. Those chemical enhancers, including polycations (e.g. polybrene, DEAE-dextran, protamine sulfate, poly-L-lysine), cationic amphipathic peptides (e.g. LAH4 derived peptide, Vectofusin-1), and Retronectin, were applied with or without physical enhancers (e.g. spinoculation). Similar approaches have also been applied for nonviral gene delivery into cells using virus-like particles (VLPs) and synthetic materials. VLPs have structures mimicking authentic viruses but without the viral genome and may carry biologic materials that are introduced to cells in the same way as viral transduction. VLPs can be used to deliver Cas9 protein and guide RNAs for gene editing (U.S. Ser. No. 10/968,253, WO2020102709, WO2021055855, US20210261957).

Retronectin (CH-296, Takara Bio) is a recombinant protein derived from human fibronectin fragments with domains that binds to integrins, VLA-4/5 and another heparin-binding domain that binds to enveloped viruses (see International Patent Application publication WO 95/26200 A1). It is often immobilized on culture dishes to be used in conjunction with spinoculation for enhancing viral transduction. It is thought this molecule brings viruses and target cells closely to facilitate interactions. Additionally, Retronectin has also been immobilized on solid microbeads for first binding viruses and subsequent targeting cells simply through gravity forces without spinoculation to assist viral transduction as for instance described in the international patent application publication WO 2010080032 A2. It is necessary to remove solid beads for therapeutic applications. As the mobility of attached materials on solid surface is limited, interaction with target cells is dependent on the surface density of attached materials. Indeed, large unilamellar vesicles (LUVs) are often used for laboratory research to mimic biological interactions on the fluid cell membrane.

In addition to taking advantage of the flexibility and disruptable nature of microbubbles in our previous disclosure (U.S. Pat. No. 10,479,976), this disclosure explores the fluid nature of cell membrane-like surface of microbubbles to enhance interaction between cells and particles for gene delivery. Instead of creating a single recombinant fusion protein with multiple functional domains (e.g. Retronectin), we are able to accomplish similar activities by placing individual small functional motifs on the fluid microbubble surface (FIG. 1). With previous (U.S. Pat. No. 10,479,976) and current disclosures, engineered microbubbles can be used for streamlining CAR-T production for cell sorting, activation, and transduction (FIG. 9). Moreover, in vivo cell-specific gene delivery is challenging. Targeted microbubbles have been used for cell-specific imaging and drug delivery in vivo. In vivo gene delivery may be enhanced by microbubbles with proper attachments.

### SUMMARY OF THE INVENTION

According to a first aspect of the invention, a method for ex vivo transduction of biomolecules from viruses, viral vectors or virus-like particles into target cells comprises: preparing a mixture by mixing a quantity of viruses, viral vectors or virus-like particles and flexible lipid shell microbubbles, said flexible lipid shell microbubbles being conjugated with one or more ligands binding to the viruses, viral vectors or virus-like particles and to the target cells; incubating the mixture over a time span allowing viruses, viral vectors or virus-like particles to bind to microbubbles; incubating the microbubbles with viruses, viral vectors or virus-like particles and the target cells to allow transduction to take place, transferring the biomolecules from viruses, viral vectors or virus-like particles into the target cells while the viruses, viral vectors or virus-like particles and the target cells are bound to the microbubbles.

According to a second aspect of the invention, flexible lipid shell microbubbles are adapted to facilitate viral transduction between viruses, viral vectors or virus-like particles and target cells, transferring biomolecules from viruses, viral vectors or virus-like particles into the target cells while the viruses, viral vectors or virus-like particles and the target cells are bound to the microbubbles, wherein the flexible lipid shell microbubbles are conjugated with bi-specific ligands that are capable of binding to both viruses, the viral vectors or virus-like particles and the target cells or are conjugated with at least a first and a second ligand differing from each other with the first ligand binding to viruses or the viral vectors but not to the target cells and the second ligand binding to the target cells but not to viruses, the viral vectors or virus-like particles.

### DETAILED DESCRIPTION OF THE INVENTION

The present invention includes microbubble-based artificial virus-presenting cells (aVPC's) with attached ligands that bind virus and target cells. As a practical application, the invention provides a process for streamlining chimeric antigen receptor T-cell (CAR-T) processing with microbubble-based T-cell selection, activation, and viral transduction. The microbubble-based artificial virus-presenting cells (aVPC's) are conjugated with attached ligands that bind virus and target cells and a process for streamlining chimeric antigen receptor T-cell (CAR-T) processing with microbubble-based T-cell selection, activation, and viral transduction. No ultrasound is delivered (sonoporation) for accomplishing viral transduction and cell activation. This method substantially increases the efficiency and reduces the amount of virus needed for gene delivery, which accounts for a main cost of gene and cell therapy products.

### BRIEF DESCRIPTION OF THE DRAWINGS

FIG. 1 illustrates a first embodiment of a microbubble according to the invention conjugated with a bispecific ligand binding to viruses and target cells.

FIG. 2 illustrates a second embodiment of a microbubble according to the invention conjugated with a first ligand binding to viruses and a second ligand binding to target cells.

FIG. 3 illustrates a third embodiment of a microbubble according to the invention conjugated with a ligand binding to viruses only.

FIG. 4 illustrates a diagram of a process according to the invention.

FIG. 5 illustrates a diagram of a functional embodiment and experimental validation of an embodiment according to the invention.

FIG. 6 illustrates the method according to the invention as divided into 3 steps of concentration (virus capture), bridging (cell binding) and searching (virus binding).

FIG. 7 illustrates a diagram of a functional embodiment and an early experimental validation of the preferred embodiment according to the invention.

FIG. 8 shows an enhanced virus-like particle delivery efficiency to CD4+ cells by microbubbles conjugated with protamine and anti-CD4 (“MB-VLP”), compared to free VLP (“VLP”) and a negative control (“medium only” without VLP).

FIG. 9 demonstrates an animal study with a bead-free system for CAR-T cell production system according to the invention.

### DETAILED DESCRIPTION OF THE DRAWINGS

The following description and drawings are illustrative and are not to be construed as limiting. Numerous specific details are described to provide a thorough understanding. However, in certain instances, well known or conventional details are not described in order to avoid obscuring the description. References to one or an embodiment in the present disclosure are not necessarily references to the same embodiment, and such references mean at least one.

The use of headings herein is merely provided for ease of reference and shall not be interpreted in any way to limit this disclosure or the following claims.

Reference in this specification to “one embodiment” or “an embodiment” or the like means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described that may be exhibited by some embodiments and not by others. Similarly, various requirements are described that may be requirements for some embodiments but not other embodiments.

The present disclosure consists of artificial virus-presenting cells (aVPC's) with attached ligands that bind viruses and target cells. It also consists of a process for streamlining chimeric antigen receptor T-Cell (CAR-T) processing with microbubble-based T-cell selection, activation, and viral transduction. The aVPC's may contain a variety of different ligand combinations that are conjugated to the microbubbles. In its preferred embodiment, the present disclosure features aVPC-containing microbubbles with two different ligands, one of which is a virus-binding ligand that does not bind to cells and one of which is a cell-binding ligand that does not bind to viruses.

Transduction of biomolecules encompasses delivery of genes or in the alternative delivery of other biomolecules that edit/regulate genes, such as siRNA, crispr-cas9 system, enzymes/proteins that may not be called “genes”. Such biological molecules can be carried by viral vectors or VLPs.

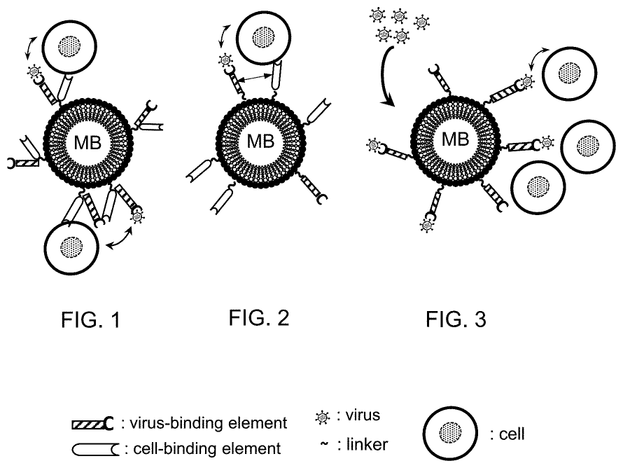

FIG. 1 shows a first preferred embodiment according to the invention. This diagram features an aVPC with a type of conjugated bispecific ligand, that allows the microbubbles to bring a virus and target cell in close proximity, either on the same or different molecules, as ligands on the lipid membrane can move freely. While binding of a cell and a virus to a single ligand is commonly illustrated in scientific and commercial documents, it is more likely that the virus and the cell bind to separate nearby ligands, as a ligand (e.g., RetroNectin protein molecule) is much smaller than a virus or cell.

FIG. 2 shows a second preferred embodiment according to the invention. This diagram features aVPC-containing microbubbles with two different ligands that bind only either the virus or the cell. This diagram features an aVPC that allows for the ratio of ligands for viruses and cells to be tuned.

FIG. 3 shows a third preferred embodiment according to the invention. The microbubbles in this embodiment with only virus-binding ligands may serve as effective aVPC's, as these microbubbles can concentrate viral particles, which leads to increased local transduction rate when microbubbles bump into cells.

Summarizing FIGS. 1-3 demonstrating microbubbles-based aVPCs, with a conjugated bispecific ligand, such as RetroNection (RN), microbubbles (MBs) can bring a virus and a target cell in close proximity, either on the same or different molecules, as ligands on the lipid membrane can move freely. Alternatively, MBs with two different ligands that bind only either the virus or the cell can also serve as aVPCs. Unlike previous conditions, the ratio of ligands for viruses and cells can be tuned. Furthermore, MBs with only virus-binding ligands may also serve as effective aVPCs, as these MBs can concentrate viral particles, which leads to increased local transduction rate when MBs bump into cells.

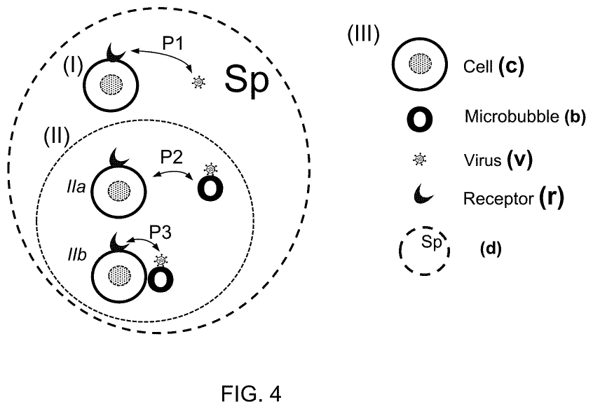

FIG. 4 shows both a structural and functional embodiment according to the invention. This aVPC embodiment is illustrated, in part, to show the substantial increase in binding probability between a virus and its receptor by restricting their interaction from 3-dimensional to 2-dimensional space. In a confined space, the binding probability between a free virus and a receptor on a cell is equivalent to the binding between a free virus and a free receptor. This concept is further illustrated in FIG. 4(I).

FIG. 4(I) shows both a structural and functional embodiment according to the invention. Here, the diameters of the cell and the microbubble are about ten and 5 micrometers respectively, and the diameters of the virus and its receptor are about 0.1 and 0.01 micrometers respectively. The items used for this interacting scheme is shown in FIG. 4(III) and consists of a cell, a microbubble, a virus, a receptor, and a confined space. The diameter of each item is expressed in the parentheses after each term.

FIG. 4(II) shows a functional embodiment according to the invention. The functional aVPC in this figure shows how when a virus is bound to a microbubble, the binding between the virus and receptor is a 2-step process. FIG. 4(II)(a) illustrates the binding between a cell and a microbubble that has abundant cell-targeting ligands compared to the diagram in FIG. 4(I). FIG. 4(II)(a) predicts a high probability of binding between its illustrated cell and microbubble. In FIG. 4(II)(b), the receptor on the cell and the virus on the microbubble are mobile because of a lipid shell. Subsequently, the searching of the virus and the receptor is on a 2-dimensional space, with probability in the figure defined as “P3.”

The ratio of the overall probability of the condition in FIG. 4(II), the product of P2 and P3, and the probability of the condition in FIG. 4(I) P1 is defined by the following formula:

(P2×P3)/P1=[(c/d)³×(b/d)³]×[(r/c)²×(v/b)²]/[(r/d)³×(v/d)³]=bc/rv (Formula 1),

wherein

P1 is the probability of binding between a free virus and a receptor on a cell, which is equivalent to the binding between a free virus and a free receptor.

P2 is the probability of binding between a cell and a microbubble (MB) that has excess cell targeting ligands.

P3 is the probability of binding between the virus on a microbubble and the receptor on the bound cell in a 2-dimensional space.

b is the diameter of the microbubble (˜5 μm).

c is the diameter of the cell (˜10 μm).

d is the diameter of the space of the container.

r is the diameter of the receptor (˜0.01 μm).

v is the diameter of the virus (˜0.1 μm).

This formula, based on physical interaction only, predicts a very substantial increase of probability for a virus binding to its target through the microbubble-based aVPC.

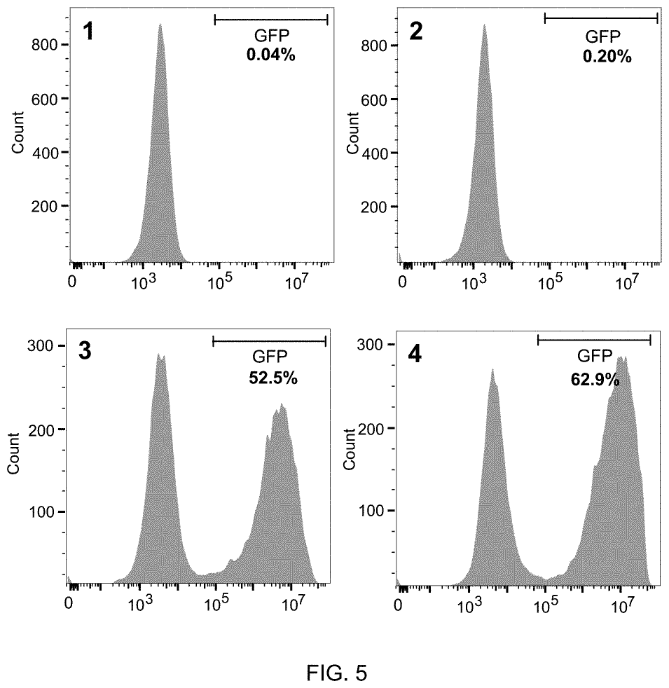

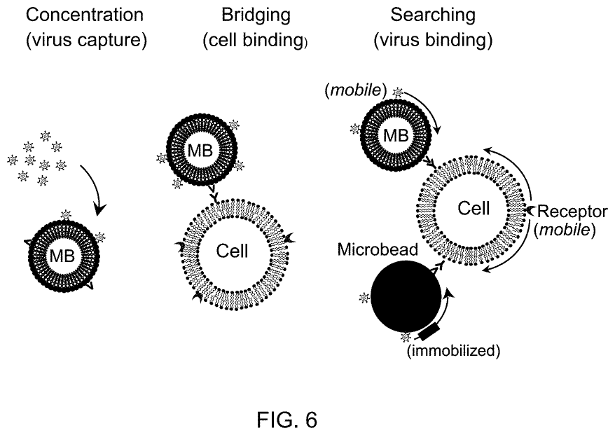

FIG. 5 demonstrates a functional embodiment and experimental validation of the embodiment illustrated in FIG. 1. This experimental validation uses recombinant retroviruses expressing green fluorescent proteins (GFP) incubated with MB-anti-CD3/CD28 treated PBMCs in the absence or presence of an equal amount of RetroNectin (RN) in various conditions. This figure's graphs show results using an experimental group as well as positive and negative controls. Recombinant retroviruses expressing GFP were incubated with MB-anti-CD3/CD28 treated PBMCs in the absence (FIG. 5(1)) or presence of RN (FIG. 5(2)). The transduction rates were poor for both of these two conditions. Conveniently, incubating RN conjugated microbubbles with viruses and PBMCs (an example of FIG. 1) (FIG. 5(4)) resulted in equivalent (or better) transduction efficiency, compared to the conventional method of using fresh RN coated plates in conjunction with 2-hour spinoculation (FIG. 5(3)). Notably, the poor transduction efficiency of using free RN (FIG. 5(2)) in solution, compared to immobilized RN (FIG. 5(3) & FIG. 5(4)), argues against the popular depiction that a virus and a cell are brought into close proximity directly by a single RN molecule.

FIG. 6 illustrates how the method according to the invention can be divided into the 3 steps: concentration (virus capture), bridging (cell binding) and searching (virus binding) and demonstrates in this connection the difference over the prior art. This it divides the molecular mechanism of how the aVPC according to the invention enhances viral transduction into the 3 steps as discussed further in the following. First, the free virus in solution will be captured by the virus-binding ligand on the microbubble (“concentration”) that will increase the chance of interaction between virus on a microbubble and its entry receptor on a cell when the microbubble and cell bind (“bridging”) through cell binding ligand on the microbubble. When ligands are immobilized on the solid surface (e.g., magnetic beads or tissue culture plate), it is obvious the density of the ligands must be at certain threshold to ensure a virus can reach its cellular entry receptor. Notably, the fluid nature of the lipid shell on a microbubble makes the “searching” process possible when sufficient time is given. Through this mechanism, the virus density on the microbubble can be reduced, compared to using vehicles made of immobile solid surface.

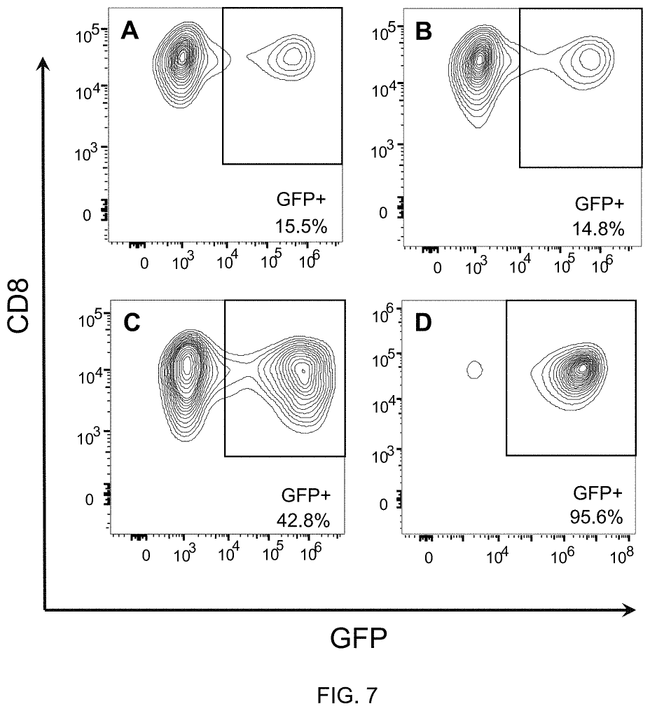

FIG. 7 shows viral transductions mediated by ligand-conjugated microbubbles, an experimental validation of the preferred embodiment according to the invention. It reduces to practice the diagram illustrated in FIG. 2. This four sub-diagrams show an experimental group, positive and negative controls, and a RetroNectin group for SupT1 cells incubated with ligand-conjugated microbubbles (MBs) for transduction with a GFP encoded gamma-retroviral vector. Only baseline transduction was detected when MBs conjugated with protamine (PRM) that binds viruses (A) or an RGD peptide (GRGDS) that binds cells (B) alone. MBs with PRM+RGD (C) significantly increased transduction efficiency in this setting. MBs conjugated with RetroNectin (D) was used as a positive control. Protamine is clinically used to bind and neutralize heparin, and the RGD peptide binds integrin receptors on cells. These two ligands were applied together to simulate the function of heparin/virus- and integrin/cell-binding domains in RetroNectin.

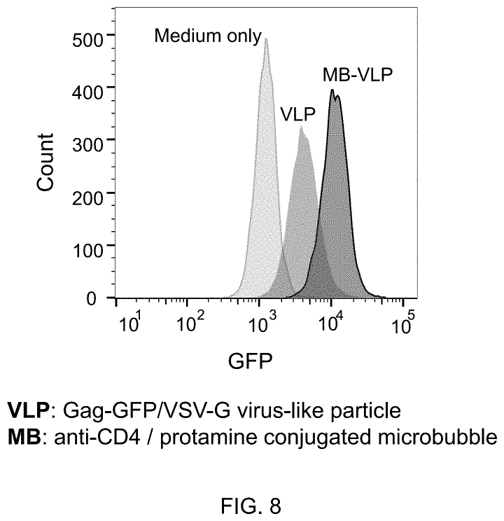

It has been well documented that a single retroviral Gag polyprotein is sufficient for self-assembly into virus-like particles. A VSV-G pseudotyped virus-like particle (VLP) was produced by co-transfecting 293-T cells with a plasmid encoding lentiviral Gag protein fused to GFP (Gag-GFP) and a second plasmid encoding VSV-G envelope protein, similar to standard lentiviral vector and VLP production, such as described in in the U.S. Pat. No. 10,968,253 (FIG. 8). A microbubble with cell binding ligand, anti-CD4 antibody and virus-binding ligand, protamine (PRM) was generated as an aVPC. The microbubble bound (labeled as “MB-VLP”) or free (labeled as “VLP”) Gag-GFP/VSV-G VLP were used to transduce SupT1 cells, which is a CD4+CD8+ T cell line. The delivery of Gag-GFP to cells was analyzed by detecting green fluorescence using flow cytometry. The result demonstrates a microbubble based aVPC enhances a VLP transduction, evident by the right shifted peak (FIG. 8, VLP vs. MB-VLP).

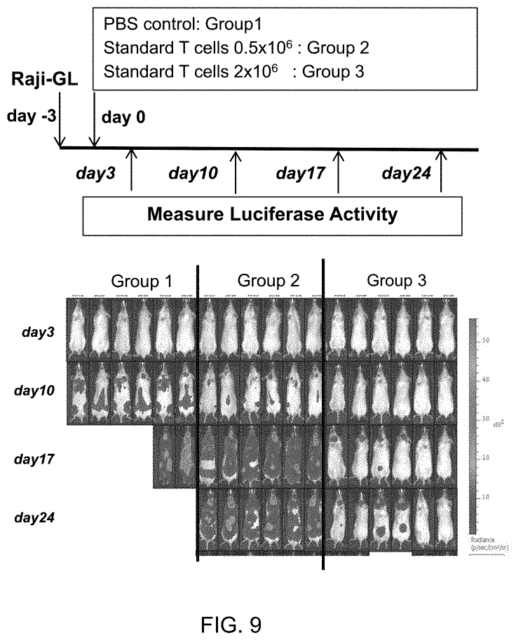

FIG. 9 demonstrates an embodiment of a bead-free system for CAR-T cell production system, peripheral blood mononuclear cells were activated with anti-CD3/CD28 microbubbles, transduced with anti-CD19 CAR retroviral vector bound to Retronectin conjugated microbubbles. Manufactured anti-CD19 CAR-T cells (two doses, 0.5 and 2 millions) or control (PBS buffer only) were injected into NSG mice bearing CD19+ Raji-GL lymphoma B cell line that expresses GFP and luciferase (FIG. 9). Bioluminescence (representing tumor burden) measurements at day 03, 10, 17, and 24 showed a dose-dependent effectiveness of CAR-T cells against Raji tumor cells in this pre-clinical animal model.

The above description of the disclosed embodiments is provided to enable any person skilled in the art to make or use the present disclosure. Various modifications to these embodiments will be readily apparent to those skilled in the art, and the generic principles described herein can be applied to other embodiments without departing from the spirit or scope according to the invention. Thus, it is to be understood that the description and drawings presented herein represent a presently preferred embodiment according to the invention and are therefore representative of the subject matter, which is broadly contemplated by the present disclosure. It is further understood that the scope according to the invention fully encompasses other embodiments that may become obvious to those skilled in the art.

This invention enables a person skilled in the art to apply microbubble-based aVPCs for transduction enhancement to virus types that are transmitted through cell-to-cell, and their derived vectors. In addition, nanoparticles with targeted ligands are like VLPs that can transfer biological molecules (e.g., proteins, nucleic acids) to specific cells. Therefore, microbubble-based aVPCs with related ligands to those targeted nanoparticles (reviewed in Lostalé-Seijo et al. 2018 Nature Reviews Chemistry. 2:258) can also enhance delivery with mechanisms described in this invention.

Further embodiments of the invention are described in the following:
Embodiment 1. A method for ex vivo transduction of biomolecules from viruses, viral vectors or virus-like particles into target cells, comprising:
preparing a mixture by mixing a quantity of viruses, viral vectors or virus-like particles and flexible lipid shell microbubbles, said flexible lipid shell microbubbles being conjugated with one or more ligands binding to the viruses, viral vectors or virus-like particles and to the target cells;
incubating the mixture over a time span allowing the viruses, viral vectors or virus-like particles to bind to microbubbles;
incubating the microbubbles with the viruses, viral vectors or virus-like particles and the target cells to allow transduction to take place, transferring the biomolecules from viruses, viral vectors or virus-like particles into the target cells while the viruses, viral vectors or virus-like particles and the target cells are bound to the microbubbles.
Embodiment 2. The method according to embodiment 1, wherein the viruses, viral vectors or virus-like particles are first bound to the microbubbles for concentrating these on the microbubbles before the target cells are introduced into the mixture for binding subsequently to the microbubbles with the viruses, viral vectors or virus-like particles already bound to the microbubbles.
Embodiment 3. The method according to embodiment 1, wherein all components of the mixture including the viruses, viral vectors or virus-like particles, microbubbles and the target cells are mixed simultaneously, allowing the viruses, viral vectors or virus-like particles and target cells to bind simultaneously to the microbubbles.
Embodiment 4. The method according to any of the preceding embodiments, further comprising bursting the microbubbles after incubation either by allowing the spontaneous bursting of the microbubbles over time, or applying pressure that is above an ambient pressure, or by adding a chemical bursting the microbubbles.
Embodiment 5. The method according to any one of the preceding embodiments, wherein the original target cell prior to preparing the mixture is a T-cell and the resulting target cell after incubation is a chimeric antigen receptor T-cell for use in a CAR-T cell therapy.
Embodiment 6. The method according to any one of the preceding embodiments, wherein the microbubbles are conjugated with bi-specific ligands that are capable of binding to both the viruses, viral vectors or virus-like particles and the target cells or are conjugated with at least a first and a second ligand differing from each other with the first ligand binding to the viruses, viral vectors or virus-like particles but not to the target cells and the second ligand binding to the target cells but not to the viruses, viral vectors or virus-like particles.
Embodiment 7. The method according to embodiment 6, wherein the target cells are T cells and the viral vectors are retroviral vectors, and the microbubbles are conjugated with protamine that binds the viral vectors and an RGD peptide that binds the target cells.
Embodiment 8. The method according to embodiment 7, wherein the target cells are CD4+ T cells, the viral vectors are replaced by virus-like particles, and the microbubbles are conjugated with protamine that binds the virus-like particles and anti-CD4 antibody that binds the target cells.
Embodiment 9. The method according to any one of the preceding embodiments, wherein the microbubbles are conjugated with retronectin as a bispecific ligand.
Embodiment 10. The method of embodiment 1, wherein the target cells include one of or a combination of T cells, B cells, tumor-infiltrating lymphocytes, dendritic cells, natural killer cells, endothelial cells, stem cells and cancer cells from human or animal blood, from other human or animal body fluids, from human or animal tissues, or from artificial buffer solutions.
Embodiment 11. The method of any one of the preceding embodiments, further comprising:
activating the target cells by adding to the mixture flexible lipid shell microbubbles conjugated to ligands capable of forming an immunological synapse with the target cells or conjugating the flexible lipid shell microbubbles being conjugated with one or more ligands binding to the viruses, viral vectors or virus-like particles and to the target cells additionally with ligands capable of forming an immunological synapse with the target cells; and
incubating the T cells with the ligands presenting flexible shell microbubbles over a time span that is sufficient for activating the sparse subset of T cells, the incubation taking place at least over a part of the incubation time simultaneously with the viral transduction taking place in the mixture.
Embodiment 12. The method of embodiment 11, wherein the target cells are T-cells and specific T-cell activation is achieved through combining with a unique peptide bound to a recombinant MHC, and anti-CD28 or with other co-stimulating molecules; and nonspecific T-cell activation is achieved through combining anti-CD3, and anti-CD28 or with other co-stimulating molecules.
Embodiment 13. The method of embodiment 12, further comprising achieving at least one of specific and nonspecific T-cell activation through combining with the co-stimulating molecules recombinant CD80 and CD86.
Embodiment 14. The method of embodiment 5, wherein the engineered T-cells expressing an anti-CD19 chimeric antigen receptor are adapted for a CAR-T cell therapy for treatment of CD19+ B cell malignancies.
Embodiment 15. Flexible lipid shell microbubbles adapted to facilitate viral transduction between viruses, viral vectors or virus-like particles and target cells, transferring biomolecules from the viruses, viral vectors or virus-like particles into the target cells while the viruses, viral vectors or virus-like particles and the target cells are bound to the microbubbles, wherein the flexible lipid shell microbubbles are conjugated with bi-specific ligands that are capable of binding to both the viruses, the viral vectors or virus-like particles and the target cells or are conjugated with at least a first and a second ligand differing from each other with the first ligand binding to the viruses or the viral vectors but not to the target cells and the second ligand binding to the target cells but not to the viruses, the viral vectors or virus-like particles.
Embodiment 16. The flexible lipid shell microbubbles of embodiment 15, wherein the ligands on the microbubbles are adapted to attach to T-cells as a target cell further adapted to bind the viruses, viral vectors or virus-like particles bringing these in close proximity to the T-cell facilitating viral transduction so that chimeric antigen receptor T-cell for use in a CAR-T cell therapy are generated by the viral transduction.
Embodiment 17. The flexible lipid shell microbubbles of one of embodiments 15-16, wherein the target cells include one of or a combination of T cells, B cells, tumor-infiltrating lymphocytes, dendritic cells, natural killer cells, endothelial cells, stem cells and cancer cells from human or animal blood, from other human or animal body fluids, from human or animal tissues, or from artificial buffer solutions.
Embodiment 18. The flexible lipid shell microbubbles of one of embodiments 15-17, wherein the microbubbles are conjugated with retronectin to increase the viruses, viral vectors or virus-like particles transduction efficiency.
Embodiment 19. The flexible lipid shell microbubbles of one of embodiments 15-18, further being conjugated to ligands capable of forming an immunological synapse with the target cells for activating and expanding the target cells.
Embodiment 20. The flexible lipid shell microbubbles of embodiment 16, further being conjugated with unique peptide bound to a recombinant MHC, and anti-CD28 or with other co-stimulating molecules for achieving specific T-cell activation.
Embodiment 21. The flexible lipid shell microbubbles of embodiment 16, further being conjugated with anti-CD3 and anti-CD28 or other co-stimulating molecules, such as recombinant CD80 and CD86 for achieving at least one of specific and nonspecific T-cell activation.

What is claimed is:

1. A method for ex vivo transduction of biomolecules from viruses, viral vectors or virus-like particles into target cells, comprising:
preparing a mixture by mixing a quantity of viruses, viral vectors or virus-like particles and flexible lipid shell microbubbles, said flexible lipid shell microbubbles being conjugated with one or more ligands binding to the viruses, viral vectors or virus-like particles and to the target cells;
incubating the mixture over a time span allowing the viruses, viral vectors or virus-like particles to bind to microbubbles;
incubating the microbubbles with the viruses, viral vectors or virus-like particles and the target cells to allow transduction to take place, transferring the biomolecules from viruses, viral vectors or virus-like particles into the target cells while the viruses, viral vectors or virus-like particles and the target cells are bound to the microbubbles.

2. The method according to claim 1, wherein the viruses, viral vectors or virus-like particles are first bound to the microbubbles for concentrating these on the microbubbles before the target cells are introduced into the mixture for binding subsequently to the microbubbles with the viruses, viral vectors or virus-like particles already bound to the microbubbles.

3. The method according to claim 1, wherein all components of the mixture including the viruses, viral vectors or virus-like particles, microbubbles and the target cells are mixed simultaneously, allowing the viruses, viral vectors or virus-like particles and target cells to bind simultaneously to the microbubbles.

4. The method according to claim 1, further comprising bursting the microbubbles after incubation either by allowing the spontaneous bursting of the microbubbles over time, or applying pressure that is above an ambient pressure, or by adding a chemical bursting the microbubbles.

5. The method according to claim 1, wherein the original target cell prior to preparing the mixture is a T-cell and the resulting target cell after incubation is a chimeric antigen receptor T-cell for use in a CAR-T cell therapy.

6. The method according to claim 1, wherein the microbubbles are conjugated with bi-specific ligands that are capable of binding to both the viruses, viral vectors or virus-like particles and the target cells or are conjugated with at least a first and a second ligand differing from each other with the first ligand binding to the viruses, viral vectors or virus-like particles but not to the target cells and the second ligand binding to the target cells but not to the viruses, viral vectors or virus-like particles.

7. The method according to claim 6, wherein the target cells are T cells and the viral vectors are retroviral vectors, and the microbubbles are conjugated with protamine that binds the viral vectors and an RGD peptide that binds the target cells.

8. The method according to claim 7, wherein the target cells are CD4+ T cells, the viral vectors are replaced by virus-like particles, and the microbubbles are conjugated with protamine that binds the virus-like particles and anti-CD4 antibody that binds the target cells.

9. The method according to claim 1, wherein the microbubbles are conjugated with retronectin as a bispecific ligand.

10. The method of claim 1, wherein the target cells include one of or a combination of T cells, B cells, tumor-infiltrating lymphocytes, dendritic cells, natural killer cells, endothelial cells, stem cells and cancer cells from human or animal blood, from other human or animal body fluids, from human or animal tissues, or from artificial buffer solutions.

11. The method of claim 1, further comprising:
activating the target cells by adding to the mixture flexible lipid shell microbubbles conjugated to ligands capable of forming an immunological synapse with the target cells or conjugating the flexible lipid shell microbubbles being conjugated with one or more ligands binding to the viruses, viral vectors or virus-like particles and to the target cells additionally with ligands capable of forming an immunological synapse with the target cells; and
incubating the T cells with the ligands presenting flexible shell microbubbles over a time span that is sufficient for activating the sparse subset of T cells, the incubation taking place at least over a part of the incubation time simultaneously with the viral transduction taking place in the mixture.

12. The method of claim 11, wherein the target cells are T-cells and specific T-cell activation is achieved through combining with a unique peptide bound to a recombinant MHC, and anti-CD28 or with other co-stimulating molecules; and nonspecific T-cell activation is achieved through combining anti-CD3, and anti-CD28 or with other co-stimulating molecules.

13. The method of claim 12, further comprising achieving at least one of specific and nonspecific T-cell activation through combining with the co-stimulating molecules recombinant CD80 and CD86.

14. The method of claim 5, wherein the engineered T-cells expressing an anti-CD19 chimeric antigen receptor are adapted for a CAR-T cell therapy for treatment of CD19+ B cell malignancies.

15. Flexible lipid shell microbubbles adapted to facilitate viral transduction between viruses, viral vectors or virus-like particles and target cells, transferring biomolecules from the viruses, viral vectors or virus-like particles into the target cells while the viruses, viral vectors or virus-like particles and the target cells are bound to the microbubbles, wherein the flexible lipid shell microbubbles are conjugated with bi-specific ligands that are capable of binding to both the viruses, the viral vectors or virus-like particles and the target cells or are conjugated with at least a first and a second ligand differing from each other with the first ligand binding to the viruses or the viral vectors but not to the target cells and the second ligand binding to the target cells but not to the viruses, the viral vectors or virus-like particles.

16. The flexible lipid shell microbubbles of claim 15, wherein the ligands on the microbubbles are adapted to attach to T-cells as a target cell further adapted to bind the viruses, viral vectors or virus-like particles bringing these in close proximity to the T-cell facilitating viral transduction so that chimeric antigen receptor T-cell for use in a CAR-T cell therapy are generated by the viral transduction.

17. The flexible lipid shell microbubbles of claim 15, wherein the target cells include one of or a combination of T cells, B cells, tumor-infiltrating lymphocytes, dendritic cells, natural killer cells, endothelial cells, stem cells and cancer cells from human or animal blood, from other human or animal body fluids, from human or animal tissues, or from artificial buffer solutions.

18. The flexible lipid shell microbubbles of claim 15, wherein the microbubbles are conjugated with retronectin to increase the viruses, viral vectors or virus-like particles transduction efficiency.

19. The flexible lipid shell microbubbles of claim 15, further being conjugated to ligands capable of forming an immunological synapse with the target cells for activating and expanding the target cells.

20. The flexible lipid shell microbubbles of claim 16, further being conjugated with unique peptide bound to a recombinant MHC, and anti-CD28 or with other co-stimulating molecules for achieving specific T-cell activation.

21. The flexible lipid shell microbubbles of claim 16, further being conjugated with anti-CD3 and anti-CD28 or other co-stimulating molecules, such as recombinant CD80 and CD86 for achieving at least one of specific and nonspecific T-cell activation.
